(19) United States
(12) Patent Application Publication
(10) Pub. No.: US 2013/0109999 A1
(43) Pub. Date: May 2, 2013

(54) Manometer for Use in Testing Spinal Region and Related Method

(71) Applicants: Neil Godara, Milton (CA); Mirvise Najafe, Richmond Hill (CA)

(72) Inventors: Neil Godara, Milton (CA); Mirvise Najafe, Richmond Hill (CA)

(73) Assignee: Kimberly-Clerk, Inc., Mississauga, ON (CA)

(21) Appl. No.: 13/285,067

(22) Filed: Oct. 31, 2011

(51) Int. Cl. A61B 5/03

(52) U.S. Cl. 600/561

(57) ABSTRACT

A manometer for measuring a differential pressure within a patient's disc includes a housing attachable to a device that is in fluid communication with the patient's disc, a pressure transducer within the housing for measuring a pressure in the patient's disc, a memory within the manometer for saving a pressure measured by the transducer, an input device on the housing for receiving input from a user to save an initial pressure measured by the transducer in the memory, and a display on the housing. The display provides an indication of an initial pressure in the patient's disc measured by the transducer before the user provides input via the input device and an indication of a differential pressure between a subsequent pressure and the initial pressure in the patient's disc after the user provides input via the input device. Related methods are also disclosed.

### TECHNICAL FIELD

The present invention relates to a device for measuring a pressure within a spinal region and a related method.

### BACKGROUND

Manometers have been proposed for measuring fluid pressures in various applications. Specialized manometers have been proposed for measuring pressures in different human body regions, including for example blood pressures and pressures within a spinal region such as a disc. Fluid pressures within a spinal region could be measured, for example, to determine the integrity of a structure there.

In some spinal procedures, a device having a lumen can be inserted into a spinal region, and an initial pressure present in the region can be measured using a manometer in communication with the device. A fluid can then be introduced into the spinal region and subsequent pressure readings can be taken. Depending on the goals and procedures, the fist and subsequent readings can provide medical personnel with insightful information about spinal region. For example, procedures might investigate how much fluid must be introduced for a patient to feel pain, at what pressure level such pain is experienced, how do pressure levels change upon introduction of a fluid or over time. Such procedures can inform the medical personnel at to whether a structure is intact, the size of a structure or an amount of damage to it, etc.

For example, International Spine Intervention Society (ISIS) Guidelines have been propagated for diagnosing discal fissures using lumbar disc stimulation via provocative discography. According to the Guidelines: A. provocation of a target disc reproduces the patient's pain, wherein provocation of adjacent disc does not reproduce pain; B. internal disruption criteria are: 1 the patient has discogenic pain, and 2. the affected disc exhibits a grade 3 or great fissure upon CT discography; C. concordant pain is the only type of pain that is diagnostic; D. manometry is used to determine the type of disc from a pressurization pain reproduction standpoint. (Patients with concordant pain at <15 psi over opening pressure have a positive disc. Those with concordant pain reproduced at 15-50 psi have a disc that is indeterminate, and those without pain until above 50 psi have a negative disc); and E. the degree of pain experienced by the patient relative to a maximum perceived pain produced during the manometric injection on a scale of 0 to 10 must be at considered to rate least 7 out of 10 (7/10).

In such situations, ISIS has developed interpretive criteria that categorize positive discography (having a positive disc) into one of 3 pain categories or an indeterminate category:
Unequivocal Discogenic Pain—stimulation of the target disc reproduces concordant pain at a level of 7/10 or greater at a pressure of less than 15 psi above opening pressures and stimulation of two adjacent discs produces no pain at all;
Definite Discogenic Pain—stimulation of the target disc reproduces concordant pain at a level of 7/10 or greater, and pain is reproduced <15 psi above opening pressure plus stimulation of one adjacent disc produces no pain or pain is reproduced at a pressure of less than 50 psi above opening pressure and stimulation of two adjacent discs does not reproduce pain at all;
Probable Discogenic Pain—stimulation of target disc reproduces concordant pain at least 7/10 at a pressure of <50 psi above opening pressure and stimulation of one adjacent disc reproduces no pain at all while another adjacent disc produces pain at greater than 50 psi but not concordant; and
Indeterminate Disc—does not meet all the above criteria yet still produces pain.

According to some protocols, injection of fluids into the disc may include use of a contrast medium. IV sedation may be administered, but the patient must be responsive during disc pressurization. A needle is advanced to the SAP then slowly across the intervertebral foramen, towards the surface of the target disc. The needle is advanced to the center of the disc (generally the center of the nucleus pulposis). Once the needle is correctly positioned in the center of the disc, its stylet is removed and the needle is connected to a system that will inject non-ionic contrast with antibiotic. As the disc is pressurized, the pressure is noted along with the patient's response. The appearance of the target disc is monitored continuously. Injection continues until: pain is produced, contrast medium escapes from the disc, the volume of injection reaches 3 ml, or firm resistance to injection is encountered. If pain is produced, the pressure and volume of injection are documented, and the patient should be asked to report the concordancy with the accustomed pain and the current intensity of pain (scale 1-10). Upon injection, contrast medium should outline the nucleus pulposis of the disc and any features of its internal structure.

Managing such a process can be complicated for the medical personnel in terms of operating the tools and obtaining the desired information. For example, surgical positioning is generally a multistep process involving identification of the site, sterilization of items for the procedure, delivery of medications before insertion of a cannula, introducer or the like. A stylet or obturator may be employed during insertion of the device connected to the manometer and then withdrawn. Other items may be inserted into the lumen of the device or attached to it to thereby place the manometer in fluid connection with the spinal region. Once the manometer is attached, typically at least several seconds to a minute or more can be required for pressures to reach equilibrium, fluids to be delivered, etc. And during all of these steps, an initial and subsequent pressures must be noted, e.g. for the determination of a differential pressure, and recorded by the medical personnel.

Accordingly, there is a continued need for improvement in devices and methods used for measuring pressures within spinal regions so as to readily and reliably obtain the desired measurements, and it would be beneficial to have a device and a method that is improved and/or overcomes some or all of the limitations of the prior art or others.

### SUMMARY

According to certain aspects of the present disclosure, a method of operating a manometer to detect a pressure differential in a patient's disc includes placing in fluid communication with the patient's disc a device that is connected to a manometer; measuring an initial pressure in the patient's disc with the manometer; displaying an indication of the initial pressure on the manometer; saving the initial pressure with the manometer; introducing a fluid into the patient's disc; measuring a subsequent pressure in the patient's disc with the manometer after introduction of the fluid; calculating a differential pressure by subtracting the initial pressure from that subsequent pressure; and displaying an indication of the differential pressure on the manometer. Various options and modifications are possible.

For example, the indication of the differential pressure may be an actual pressure. Also, the indication of the differential pressure could be an indication of a range corresponding to a physiological condition within a group of such ranges.

If desired, the fluid may be introduced into the patient's disc until the patient detects an amount of pain at which point the subsequent pressure is measured.

The initial pressure may be saved responsive to the pressing of a button on the manometer.

According to certain other aspects of the disclosure, a manometer for measuring a differential pressure within a patient's disc includes: a housing attached to a device that is in fluid communication with the patient's disc, a pressure transducer within the housing for measuring a pressure in the patient's disc, a memory within the manometer for saving a pressure measured by the transducer, an input device on the housing for receiving input from a user to save an initial pressure measured by the transducer in the memory, and a display on the housing. The display provides an indication of an initial pressure in the patient's disc measured by the transducer before the user provides input via the input device and an indication of a differential pressure between a current pressure after the user provides input via the input device. Various indications of the display are possible.

For example, the indication of the differential pressure may be in pressure units, and/or the indication of the differential pressure may be an indication of a range corresponding to a physiological condition within a group of such ranges. The differential pressure may correspond to a pressure in the patient's disc after fluid is introduced to the patient's disc via the device whereby the patient detects an amount of pain. The input device may be a button and initial pressure may be saved responsive to the pressing of the button by the user. The display may show the differential pressure rather than an actual pressure after the button is pressed by the user.

These features and others will become apparent in the detailed description that follows.

### BRIEF DESCRIPTION OF THE DRAWINGS

In order that the invention may be readily understood, embodiments of the invention are illustrated by way of examples in the accompanying drawings, in which:

FIG. 1 is a perspective view of a device for measuring a pressure within a spinal region according to certain aspects of the present disclosure;

FIG. 2 is a front view of the device of FIG. 1;

FIG. 3 is a side view of the device of FIG. 1;

FIG. 4 is a side view as in FIG. 3 partially broken away, showing internal communication between manometer components, syringe and tubing elements;

FIG. 5 is a front view of a manometer portion of the device of FIG. 1 showing display elements and buttons; and

FIGS. 6 through 11 show the front view of the manometer portion as in FIG. 5, illustrating a series of steps and resulting displayed information that could occur during a use of the device of FIG. 1 in a given procedure.

### DETAILED DESCRIPTION

With specific reference now to the drawings in detail, the particulars shown are by way of example and for purposes of illustrative discussion of some embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For the purposes of this invention, proximal generally indicates that portion of a device or system next to or nearer to a user (when the device is in use), while the term distal generally indicates a portion further away from the user (when the device is in use).

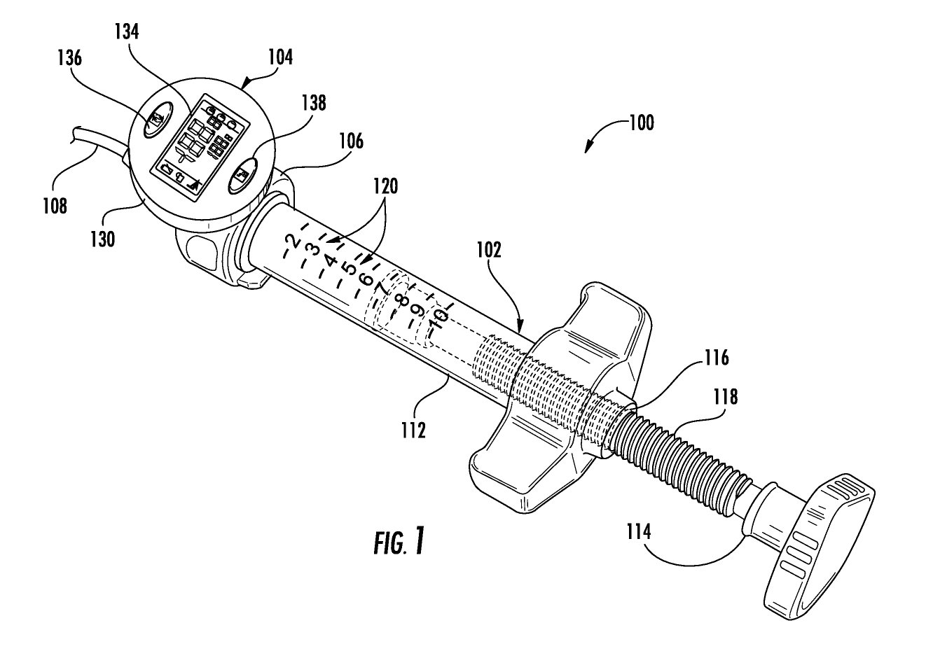

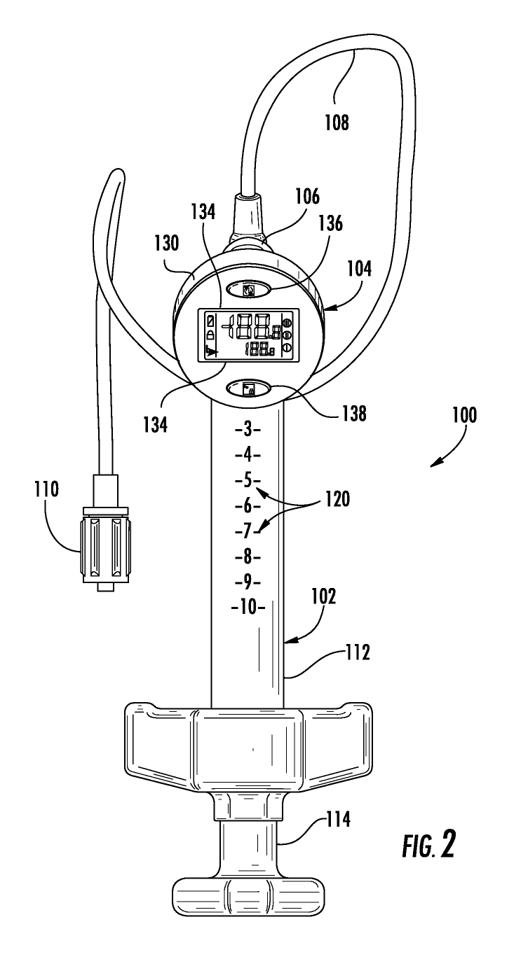

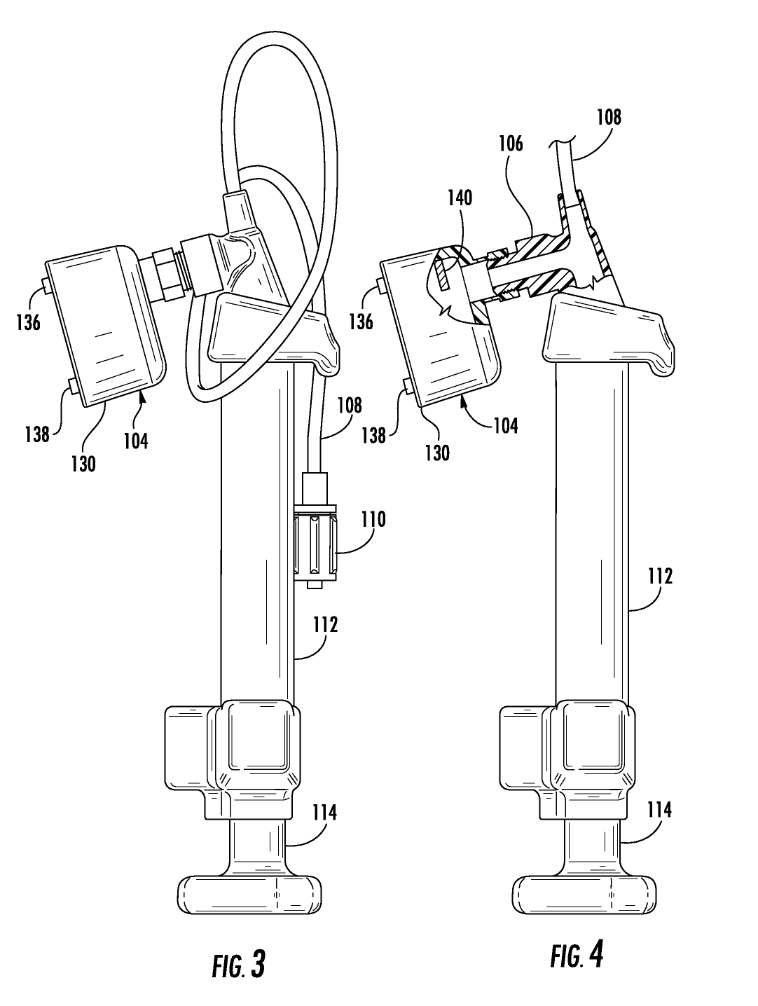

FIGS. 1-5 show one example of a device 100 for taking a measurement from a spinal region. Device 100 includes a syringe portion 102 and a manometer portion 104 attached to the syringe portion by an adaptor 106. Flexible tubing 108 is attached to an end of syringe portion 102 and leads to an adaptor 110 for attachment to elements to be placed into a spinal region.

Syringe portion 102 includes a tube 112 and plunger 114, and may have mating threads 116,118 for position adjustment. Tube 112 may include markings 120 thereon at locations indicating volume when plunger 114 is moved, and a locking lever (not shown) may be provided to set the position of plunger 114 within tube 112. The syringe portion 102 may have a capacity of for example 10 cc or 20 cc. Various syringes having such flexible tubing adapted for receiving a manometer and having the features described above or others can be obtained from Atrion Medical Products, as well as other companies. Accordingly, no limitation on the types of syringes that can be used according to the present invention should be understood to exist.

Manometer 104 includes a housing 130 with a display 134, input devices 136, 138 such as buttons, and an attachment portion for connecting to syringe portion 102, for example via adaptor 106. Display 134 may comprise an LCD screen, and/or LED's, lights, or any other type or number of indicator. Specific indications and functions provided by display 134 will be described below.

Internally, manometer 104 includes a pressure transducer 140 schematically shown) in communication with the interior of the syringe portion and tubing so as to be able to measure a pressure in the spinal region. Manometer 104 may also include conventional electronic elements (not shown) such as executable code stored in a memory, such as on chip or a solid state device, and a conventional power source, such as a battery or a connection to an external power source. Manometer 106 therefore has the ability to receive input from a user, to provide output to a user, and to have the user change states and functions by using input and output devices. Manometer 106 may have external connections (not shown) to power the device and/or to provide input/output communication regarding measured parameters or the like.

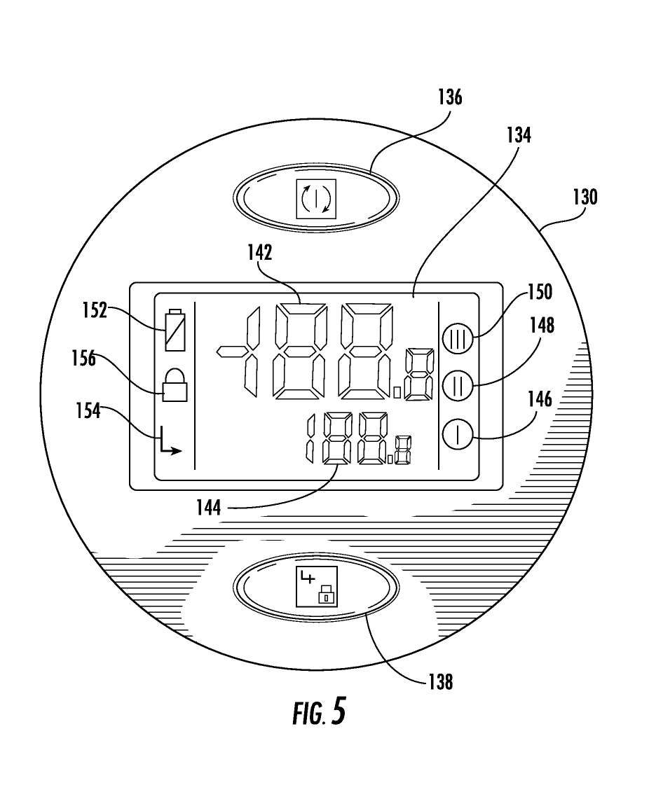

As shown in FIG. 5, manometer 104 includes buttons 136, 138. The buttons may be mechanical, touch screen elements, etc., as desired. Button 136 is used to start use or clear saved information. Button 138 is used to save information. If desired, each button could have related indicia thereon to assist the user in recognizing such functions. Other button arrangements and numbers could be used as well.

Display 134 includes two numerical fields 142 and 144 which may be used to provide pressure information (for example in psi or other units). One such field could be used for an initial pressure reading and one for another reading (e.g., gross, accumulative, differential, or subsequent pressure).

Display 134 also may include indications of pressure levels not in units but instead corresponding to diagnostic levels. For example, in certain procedures (as described below) it may be important to know if a differential pressure is below or above a certain number, or within a certain range. Therefore, display includes elements 146,148,150 that indicate multiple ranges. For example, range I may indicate a differential pressure from 0-15 psi, range II from 15-50, and range III over 50. Such ranges may correspond to a level of physical structural integrity of the spinal region upon introduction of fluid from the syringe into the spinal region. Of course, various other levels, ranges and numbers of indicators could be employed. Also, the ranges could be set to any desired values by the user using buttons 135, 138 or others.

Display 134 may also include an indicator as to battery condition 152, an indicator as to whether a pressure level has been saved 154, and an indicator as to whether a lock feature has been engaged 156. Button 136 or 138 may function as on/off button (for example, by a long press and hold, simultaneous press and hold, etc.), or a separate power button (not shown) may be provided.

The device may be operated as follows, whether or not following above ISIS Guidelines, although it should be understood that various other methods of use are possible. First, the user attaches appropriate equipment together, places the needle in the patient's disc, etc. as described above. The user then energizes or powers the manometer using one or more of buttons 136,138 or a power switch. If desired, power may be turned on by pressing or “long-pressing” button 136. LCD 134 and any other display elements are then powered on. If the device is already on and a second use to being made, pressing a button such as button 136 could be used to clear all data and ready the manometer for use.

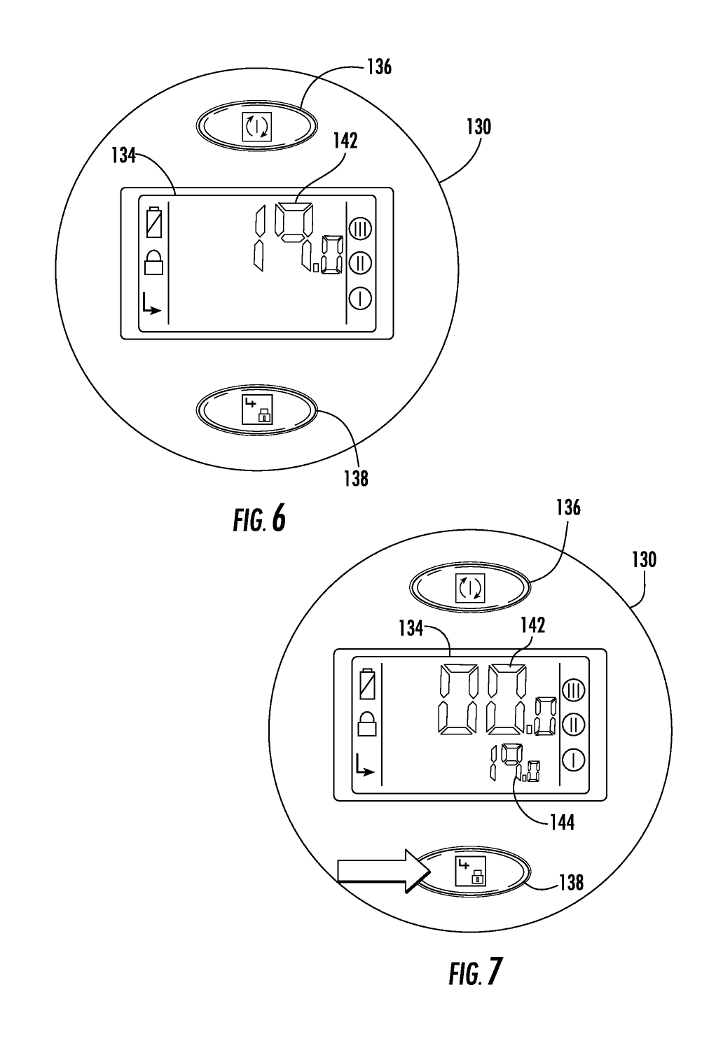

As shown in FIG. 6, a pressure reading will show in a display field such as numerical field 142. Such pressure will be the pressure within the patient's disc, and may be displayed in psi or any other desired units. When the pressure is stabilized and/or the user is ready to begin administering fluid, a button such as button 138 is pressed to store the detected pressure as an initial pressure in a memory storage location as shown in FIG. 7. After button 138 is pressed, field 144 is activated (previously it could have shown 0.00 psi or a duplicate of what was in field 142, or have been blank, as desired). If desired after activation, field 144 could be showing the stored initial pressure and field 142 could be showing the pressure being detected as fluid is injected by manipulation of the syringe. Preferably, as shown in FIG. 8, field 142 shows a differential reading from the initial pressure.

Simultaneously, if one or more elements 146,148,150 are provided to indicate ranges, the relevant range element can be featured, illuminated or display (with others not being so treated). For example, 146 (range I) can display while the differential pressure is between 0 and 15 psi, 148 (range II) while between 15-50 psi and 150 (range III) for differential pressure above 50 psi. Alternatively, I, II and III may be continuously displayed, with a related signalling indication being activated adjacent each element as pressure changes. FIG. 8 shows range I being featured, indicating that the measured pressure of 13.0 psi is in the range of 0-15 psi.

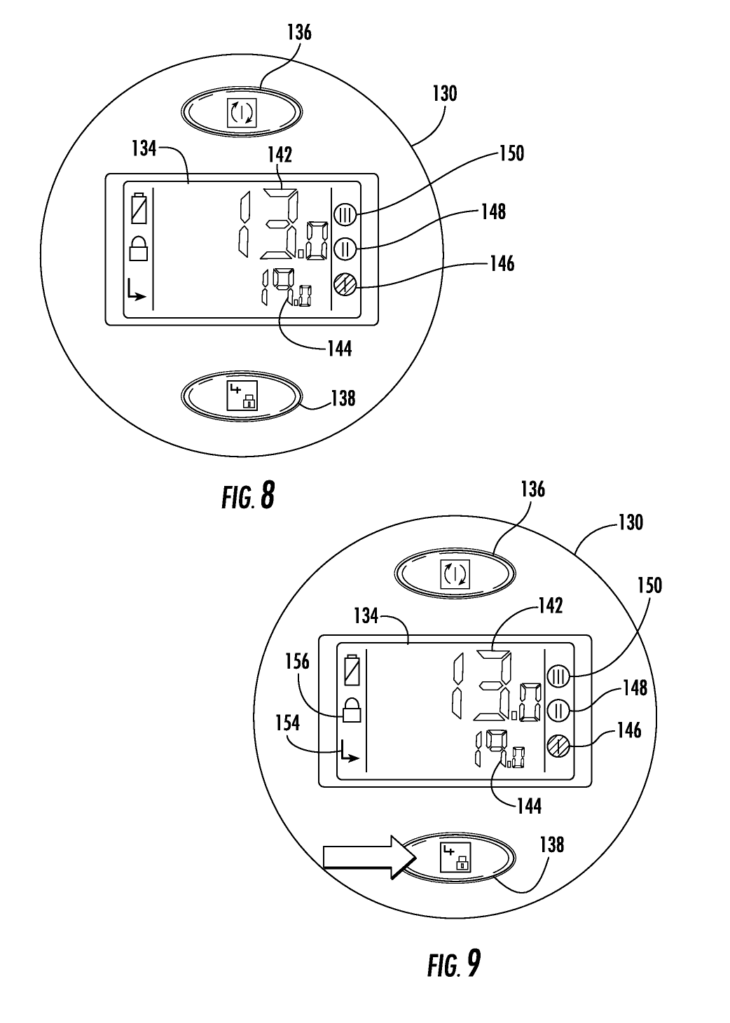

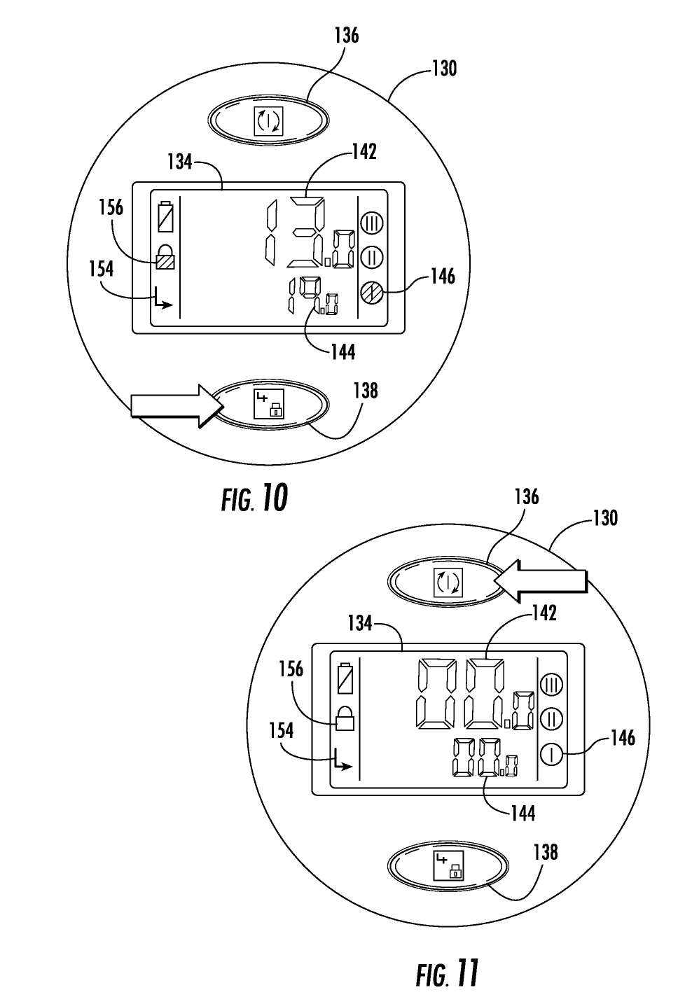

When an event occurs that prompts the user to record the differential pressure, the user can manipulate a button again, such as button 138, to save the currently displayed pressure, as shown in FIG. 9. This could occur for example, if the patient indicates a degree of pain or if contrast fluid leakage is visualized to a certain extent. At that point fields 142 and 144 of display 134 can be showing the initial pressure and a subsequent (likely differential) pressure. Elements 154 and 156 can provide information as to stored and locked information and functionality status. Readings of display fields can be stored or locked, for example, by pressing or prolonged pressing a button such as button 138, as shown in FIG. 10. The process can be restarted by pressing a button, such a button 136, prolonged pressing a button, etc., as shown in FIG. 11, thereby clearing the indications provided by elements 142,144, 146, 148, 150, 154 and 156. For clarity, various of the display elements could be selectively activated when desired. For example, elements 152, 154 and 156 could be hidden unless a button is displayed related to their functionality, or if battery levels drop.

The user thus has ready access to measured pressure readings and can be directing most attention to the patient, other equipment, visualizing contrast fluid on a display, etc., rather than watching a needle on an analog device. Use of buttons 136, 138 provides tactile feedback requiring less visual attention, although it should be understood other arrangements and input devices could be used as buttons. The user therefore can be more attentive to the patient and other elements and the manometer does the work of measuring, storing and displaying pressures, relieving the medical personnel from having to do so and thereby providing a ready and more reliable system than presently available.

As a great benefit to the user, the user can operate the device using a button for start and for save, using a simple tactile feel while directing attention to the patient or areas other than the face of the manometer. Important information is displayed or shared depending on user choice, greatly simplifying procedures for the user.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

We claim:

1. A method to detect a pressure in a patient's disc comprising:
placing a device in fluid communication with the patient's disc;
measuring an initial pressure in the patient's disc with a manometer;
displaying an indication of the initial pressure on the manometer;
saving the initial pressure within the manometer;
introducing a fluid into the patient's disc;
measuring a subsequent pressure in the patient's disc with the manometer after introduction of the fluid;
calculating within the manometer a differential pressure from the initial pressure and the subsequent pressure; and
displaying an indication of the differential pressure on the manometer.

2. The method of claim 1, wherein the indication of the differential pressure is the subsequent pressure less the initial pressure.

3. The method of claim 1, wherein the indication of the differential pressure is an indication of a range corresponding to a physiological condition within a group of such ranges.

4. The method of claim 1, wherein the fluid is introduced into the patient's disc until the patient detects an amount of pain at which point the subsequent pressure is measured.

5. The method of claim 1, wherein the initial pressure is saved responsive to the pressing of a button on the manometer.

6. A manometer for measuring a differential pressure within a patient's disc comprising:
a housing attachable to a device in fluid communication with the patient's disc;
a pressure transducer within the housing for measuring a pressure in the patient's disc;
a memory within the manometer for saving a pressure measured by the transducer;
an input device on the housing for receiving input from a user to save an initial pressure measured by the transducer in the memory; and
a display on the housing for displaying an indication of a pressure in the patient's disc measured by the transducer before the user provides input via the input device and for displaying an indication of another pressure in the patient's disc after the user provides input via the input device.

7. The manometer of claim 6, wherein the indication of pressure includes a differential pressure as determined from a pressure in the patient's disc after the user provides input via the input device less the initial pressure.

8. The manometer of claim 7, wherein the indication of the differential pressure is an indication of a range corresponding to a physiological condition within a group of such ranges.

9. The manometer of claim 7, wherein the differential pressure corresponds to a pressure in the patient's disc after fluid is introduced to the patient's disc via the device whereby the patient detects an amount of pain.

10. The manometer of claim 6, wherein the input device is a button.
